(19) United States
(12) Patent Application Publication
(10) Pub. No.: US 2013/0218149 A1
(43) Pub. Date: Aug. 22, 2013

(54) CRYO-THERAPY SPRAY DEVICE

(71) Applicants: Ori Braun, Palo Alto, CA (US); Gavriel J. Iddan, Haifa (IL); Eyal Kochavi, Haifa (IL); Elisha Rabinovitz, Haifa (IL); Zvika Gilad, Haifa (IL)

(72) Inventors: Ori Braun, Palo Alto, CA (US); Gavriel J. Iddan, Haifa (IL); Eyal Kochavi, Haifa (IL); Elisha Rabinovitz, Haifa (IL); Zvika Gilad, Haifa (IL)

(73) Assignee: GIVEN IMAGING LTD., Yoqneam (IL)

(21) Appl. No.: 13/809,050

(22) PCT Filed: Jul. 7, 2011
(86) PCT No.: PCT/US11/43161

§ 371 Date: Apr. 9, 2013

Related U.S. Application Data
(60) Provisional application No. 61/362,625, filed on Jul. 8, 2010.
(60) Provisional application No. 61/365,676, filed on Jul. 19, 2010.

(51) Int. Cl. A61B 18/02

(52) U.S. Cl. CPC A61B 18/02
USPC 606/21; 606/22

(57) ABSTRACT

A device for cryotherapy treatment of gastrointestinal lesions includes a cooling member that may be attached to a first tube for pressurizing cryogenic fluid through the tube and into the cooling member through nozzles located at the distal end of the first tube. A second tube may be attached to the cooling member for evacuating the cryogenic fluid from within the cooling member, following the fluid's expansion once it exits the first tube. The cryotherapy device may be attached to an endoscope such that the first tube may be passed through the endoscope's working channel, while the second tube may be passed along the endoscope's circumference. The cryotherapy device may further comprise securing means attached to the first tube, for securing the first tube to the endoscope's working channel, thus preventing free rotation of the cryotherapy device within the endoscope, relative to the rotation of the endoscope. In addition, the securing means assist in maintaining a constant and known location of the nozzles relative to the distal end of the endoscope.

### FIELD OF THE INVENTION

The present invention is related to the field of cryosurgery or cryotherapy devices, and more specifically to cryotherapy devices for treating gastrointestinal (GI) diseases.

### BACKGROUND OF THE INVENTION

Cryosurgery or cryotherapy is a technique by which undesired lesions are destroyed by freezing. Tissue destruction due to freezing includes direct injury to cells caused by ice crystal formation, as well as delayed injury.

There are a few known cryotherapy devices that are inserted into the gastrointestinal (GI) tract while attached to an endoscope. However, in those devices, the field of view of the endoscope's imager is typically obstructed by the cryotherapy device. In addition, in order for the freezing to be effective, a low temperature should be sustained at the treated area for a few minutes or even a fraction of a minute. In many known cryotherapy devices, cryogenic fluid (coolant) flows and expands through a nozzle of a small diameter, and the large pressure difference between the cryogenic fluid's pressure and the surroundings' pressure leads to a change in temperature, typically causing the cryogenic fluid to lower its temperature. However, in order to maintain a large pressure difference between the cryogenic fluid and its surroundings and thus avoid backpressure, which may reduce the cooling effect, there must be evacuation of expanded cryogenic fluid subsequent to it freezing an area of interest.

In some known devices, the coolant's evacuation is done through a tube that passes through the endoscope's working channel. Since the evacuation tube passes through the endoscope's working channel, the tube's cross section area is restricted by the diameter of the endoscope's working channel. Therefore, in such devices, evacuation is limited, i.e., sustaining low temperature for efficient freezing is limited, or the use of such cryotherapy devices is limited to be used with only large diameter endoscopes that are not of standard size and are not commonly practiced. Furthermore, when evacuation is limited as is in known cryotherapy devices, cryogenic fluid may not be efficiently evacuated, thus the fluid (typically gas) may penetrate into a different GI region and inflate it, which might harm that region. For example, when esophageal lesions are treated with cryosurgery, fluid that is not efficiently evacuated from the esophagus might enter the stomach and inflate it, which might lead to stomach perforation. In addition, in cryotherapy devices that include use of a cryogenic fluid jet, wherein the cryogenic fluid or coolant exits through a nozzle and is directly applied onto the tissue in the form of a spray, when the operator manipulates the endoscope in order to try to direct the cryotherapy device to a specific area of interest, the cryotherapy device freely rotates within the endoscope, relative to the rotation of the endoscope, thus making it difficult on the operator to control the direction of the jet, which might then freeze an area different than the area of interest. In addition, the distance of the nozzle from the lesion is not constant when the cryotherapy catheter is not fixed to the endoscope. As a result, the treatment outcome is not predictable, and it is difficult for the operator to follow a protocol of cryosurgery.

Therefore, there is a need for a modified cryotherapy device which would allow imaging during the procedure of freezing the tissue, which would sufficiently maintain a low temperature for the minimum required period of time and which would enable easy manipulation of the cryotherapy device towards a lesion with respect to the endoscope through which it passes.

### SUMMARY OF THE INVENTION

The present invention provides devices and systems for cryotherapy, which may be inserted through an endoscope.

According to some embodiments of the present invention, the cryotherapy device, which is inserted through an endoscope, may be inserted through the distal end of the endoscope, i.e., the end that is farther away from the proximal end that the operator holds when maneuvering the endoscope. The cryotherapy device may be inserted through the endoscope's distal end and may pass through the working channel of the endoscope. According to some embodiments, part of the cryotherapy device may pass through the working channel, while part of the device may pass along the circumference of the endoscope (i.e., the endoscope's outer wall).

In some embodiments, both parts are inserted via the endoscope's distal end. If the cryotherapy device was to be inserted via the endoscope's proximal end, similarly to other standard devices, the cryotherapy device's distal end, which is to be in direct contact with a tissue to be treated and which may cause that tissue to freeze, might have been too large for passing through the endoscope, and would have to be connected to the rest of the cryotherapy device through connecting means, e.g., screws or other known coupling means. Such connecting means might not withstand the high pressure of a coolant that would pass through them during the freezing procedure. By inserting the cryotherapy device via the endoscope's distal end, there is no need for connecting means between the device's cooling distal end and its high pressure and evacuation tubes.

According to some embodiments, the high pressure tube through which the coolant is introduced into the lumen or through which the coolant is brought in close proximity to the tissue may be passed through the endoscope's working channel. However, one or more evacuation tubes, through which expanded fluid may be evacuated to outside the lumen, may pass along the endoscope's circumference, thus not limiting the evacuation tube's diameter to the working channel's diameter, and enabling more volume of fluid to be evacuated from the lumen, thereby sustaining low temperature around the treated tissue more easily.

According to some embodiments, the cryotherapy device may enable observation of the treated areas during the cryotherapy procedure. In some embodiments, the cryotherapy device does not block the imaging unit from acquiring images of the area to be treated, as well as of the cryotherapy device during operation.

According to some embodiments, the cryotherapy device may be forced to rotate with the endoscope as one unit, which makes it easier on the operator to control movement and rotation of the cryotherapy device so as to treat a specific area of interest.

According to some embodiments, the cryotherapy device may comprise a rotatable component located at the distal end of the device. The rotatable component may be forced to rotate around a longitudinal axis of the cryotherapy device, by the force of the fluid being pushed through the device. A free spin of the distal end of the cryotherapy device may enable peripheral treatment of tissue that surrounds the distal end of the cryotherapy device.

### BRIEF DESCRIPTION OF THE DRAWINGS

The above and other objects and advantages of the invention will be apparent upon consideration of the following detailed description, taken in conjunction with the accompanying drawings, in which the reference characters refer to like parts throughout and in which:

FIGS. 1A and 1B illustrate schematic lengthwise sectional views of a cryotherapy device's distal end before and after insertion through an endoscope's distal end, respectively, in accordance with one embodiment of the present invention;

FIG. 1C illustrates a schematic lengthwise sectional view of the cryotherapy device of FIGS. 1A-B after inflation, in accordance with an embodiment of the present invention;

FIG. 1D illustrates a schematic upper view of the cryotherapy device of FIGS. 1A-C, in accordance with one embodiment of the present invention;

FIG. 2 schematically illustrates a cryotherapy system in accordance with one embodiment of the present invention;

FIGS. 3A and 3B illustrate schematic lengthwise sectional views of a cryotherapy device's distal end before and during insertion through an endoscope's distal end, respectively, in accordance with one embodiment of the present invention;

FIGS. 4A and 4B illustrate schematic lengthwise sectional views of a cryotherapy device's distal end before and during insertion through an endoscope's distal end, respectively, in accordance with another embodiment of the present invention;

FIGS. 5A and 5B illustrate schematic lengthwise sectional views of a cryotherapy device's distal end before and during insertion through an endoscope's distal end, respectively, in accordance with yet another embodiment of the present invention;

FIGS. 6A and 6B illustrate schematic lengthwise sectional views of a cryotherapy device before and after immobilization to the lumen, respectively, in accordance with one embodiment of the present invention;

FIGS. 7A and 7B illustrate schematic lengthwise sectional views of a cryotherapy device before and after immobilization to the lumen, respectively, in accordance with another embodiment of the present invention;

FIGS. 8A and 8B illustrate schematic lengthwise sectional views of a cryotherapy device in accordance with one embodiment of the present invention;

FIG. 9 illustrates an end view of a cryotherapy device in accordance with another embodiment of the present invention;

FIG. 10 illustrates a schematic lengthwise sectional view of a cryotherapy device's distal end in accordance with one embodiment of the present invention;

FIG. 11 is a schematic illustration of a cryotherapy system in accordance with one embodiment of the present invention;

FIGS. 12A and 12B illustrate schematic lengthwise sectional views of a cryotherapy device's distal end before and after insertion through an endoscope's distal end, respectively, in accordance with one embodiment of the present invention;

FIG. 12C illustrates a schematic upper view of the cryotherapy device of FIGS. 12A-B, in accordance with one embodiment of the present invention;

FIGS. 13A and 13B illustrate schematic lengthwise sectional views of a cryotherapy device's distal end before and after insertion through an endoscope's distal end, respectively, in accordance with another embodiment of the present invention;

FIG. 13C illustrates a schematic upper view of the cryotherapy device of FIGS. 13A-B, in accordance with an embodiment of the present invention;

FIG. 14 illustrates a location along the endoscope at which are located securing means for attaching a cryotherapy device to an endoscope in accordance with one embodiment of the present invention;

FIGS. 15A and 15B illustrate schematic lengthwise sectional views of two attachment mechanisms for attaching a cryotherapy device to an endoscope, in accordance with other embodiments of the present invention;

FIG. 16A illustrates a schematic lengthwise sectional view of a cryotherapy device, in accordance with one embodiment of the present invention;

FIGS. 16B and 16C illustrate cross sectional and schematic end views of components within and outside the cryotherapy device of FIG. 16A, respectively, in accordance with one embodiment of the present invention;

FIG. 17 illustrates a schematic lengthwise sectional view of a cryotherapy device within a lumen, in accordance with one embodiment of the present invention;

FIG. 18A illustrates a schematic lengthwise sectional view of a cryotherapy device, in accordance with another embodiment of the present invention;

FIG. 18B illustrates a schematic cross-sectional view of a component within the cryotherapy device of FIG. 18A, in accordance with an embodiment of the present invention; and

FIGS. 19A and 19B illustrate schematic lengthwise sectional views of a cryotherapy device, before and during operation, respectively, in accordance with an embodiment of the present invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE DRAWINGS

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

The cryotherapy devices described below are a modification of the current cryotherapy devices as known today. The cryotherapy devices described in the present invention enable imaging an area to be treated during the cryo-ablation procedure, as well as imaging the cryotherapy device during operation, a feature which is not achievable in current cryotherapy devices. In addition, the cryotherapy devices according to the present invention ensure effective cooling of fluid prior to treating an area of interest, as well as effective evacuation of expanded fluid from within the cryotherapy device or from within the lumen to outside the lumen, thus maintaining a low temperature for a sufficient period of time, which is necessary for performing a successful cryosurgical treatment.

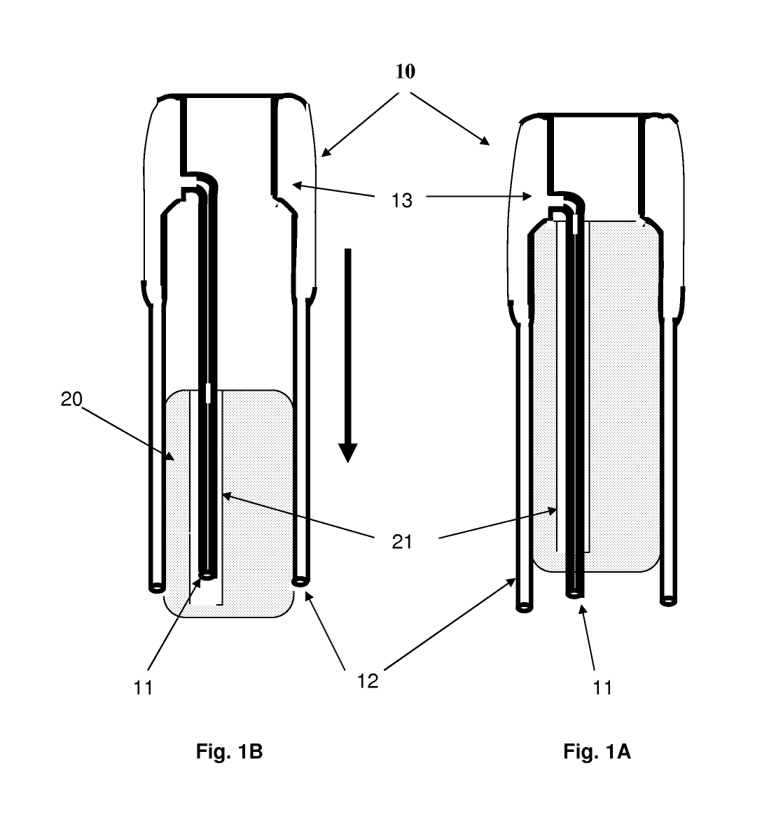

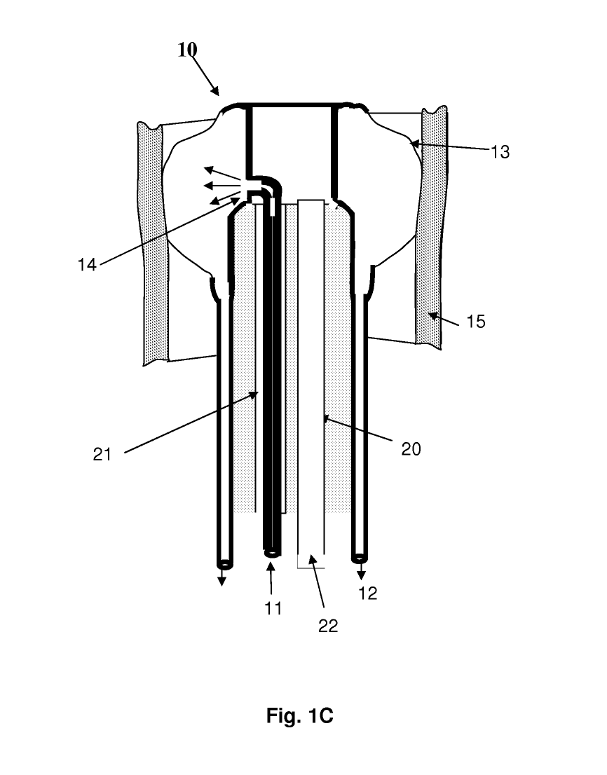

Reference is now made to FIGS. 1A and 1C. FIGS. 1A and 1B illustrate schematic lengthwise sectional views of a cryotherapy device's distal end before and after insertion through an endoscope's distal end, respectively, in accordance with one embodiment of the present invention. FIG. 1C illustrates a schematic lengthwise sectional view of the cryotherapy device of FIGS. 1A-B after inflation, in accordance with an embodiment of the present invention.

FIG. 1A illustrates a cryotherapy device 10, which may comprise a high pressure tube 11 through which a coolant (or cryogenic fluid) may be pressurized. In some embodiments, the high pressure tube 11 may have an opening or nozzle of a very small diameter through which the pressurized fluid exits the tube 11. When fluid exits the tube through the nozzle, the fluid's high pressure decreases dramatically, while undergoing pressure balance with the environment's low pressure, which causes Joule-Thomson effect, i.e., the change in fluid pressure is accompanied by a change in fluid temperature to a typically lower and more suitable temperature for tissue treatment.

Cryotherapy device 10 may further comprise a low pressure tube or evacuation tube 12, through which the fluid that has expanded following its exit through pressurized tube 11 may be evacuated to outside of the lumen, in order to maintain a low temperature at the area of interest. According to some embodiments, evacuation tube 12 may be used to vent out the coolant after its expansion by connecting tube 12 to a vacuum setup. In other embodiments, evacuation tube 12 need not be connected to an “active” suction setup, e.g., a vacuum setup, thus enabling “passive” fluid evacuation to take place by pressure differences. The coolant's pressure is higher than the approximately atmospheric pressure present within evacuation tube 12. Thus, in order to overcome the pressure difference, the high pressurized cryogenic fluid would move into lower pressure tube 12 where there is lower pressure, and out of the cryotherapy device.

According to some embodiments, cryotherapy device 10 may comprise a cooling member, e.g., balloon 13, which may be inflated by insertion of coolant into it (FIG. 1C). When cryogenic fluid is pressurized through tube 11 and into balloon 13, balloon 13 may be inflated as well as be cooled down. In some embodiments, a balloon 13 has a symmetrical shape and may enable symmetrical peripheral cryosurgical treatment, typically at cylindrically shaped lumens, e.g., the esophagus, and the small bowel. Using a balloon such as balloon 13 may assist in freezing an entire tissue that surrounds balloon 13 and that is in contact with balloon 13 during a relatively short period of time, instead of freezing different areas of the tissue at different time periods.

Balloon 13 may be made of an expandable material, e.g., latex, bio-grade polyurethane, Polyethylene terephthalate (PET) or nylon elastomers. According to some embodiments, balloon 13 may be made of polymers that are able to expand up to a certain fixed size, while according to other embodiments the balloon size may be adjustable such that balloon 13 may not be substantially limited in volume of expansion. Balloon 13 may typically have a thin wall in order for the cryogen to quickly cool it, thus causing tissue that is in contact with the balloon to freeze.

As can be seen in FIG. 1B, the cryotherapy device 10 may be inserted into an endoscope 20 via the distal end of endoscope 20. In some embodiments, the high pressure tube 11 may be passed through the endoscope's working channel 21, thus tube 11 should be of a smaller diameter than that of the endoscope's working channel 21. Evacuation tube 12, which should typically be of a larger diameter than the high pressure tube, in order to evacuate fluid at low pressure, may be passed along the external shell of endoscope 20, e.g., the endoscope's circumference, thus not being limited by the working channel's diameter.

FIG. 1C illustrates cryotherapy device 10 after it is inserted through the distal end of endoscope 20, while high pressure tube 11 may be passed through the working channel 21 of endoscope 20, and evacuation tube 12 may be passed along the external endoscope's shell. In FIG. 1C, balloon 13 may be inflated by fluid being pressurized through high pressure tube 11 and out of cold fluid inflation port 14 into the space of balloon 13.

Reference is now made to FIG. 1D, which illustrates a schematic upper view of the cryotherapy device of FIGS. 1A-C, in accordance with one embodiment of the present invention. According to some embodiments, balloon 13 may be designed such that the endoscope's 20 imaging channel 22 is not obscured but rather able to acquire images of the lumen, while the balloon 13 is used to freeze the tissue with which it is in contact. In addition, balloon 13 is designed so as to not obscure the illumination channel 23, thus allowing light to impinge onto the lumen wall, and to later reflect onto the imager that is within imaging channel 22.

In some embodiments, balloon 13 may be positioned on the circumference of endoscope 20 and may be forced to expand only in radial directions farther away from the endoscope's 20 longitudinal axis, i.e., balloon 13 may be forced to expand from the external endoscope 20 shell and outwards (and to not expand inwards, closer to endoscope 20 longitudinal axis), thus leaving the imaging unit 22 and illuminating unit 23 unblocked. Balloon 13 may be forced to expand only in radial directions farther from endoscope 20 longitudinal axis, by for example, having a thick wall at the sides of balloon 13 that are closer to the longitudinal axis, i.e., at the inner balloon walls 13b, while having a thin wall at the sides of balloon 13 that are farther away from the endoscope's longitudinal axis, i.e., at the outer balloon walls 13a. Other ways of forcing expansion of balloon 13 in certain directions may be used.

In some embodiments, as illustrated in FIG. 1C, balloon 13 may protrude from the distal end of the endoscope 20. In such embodiments, balloon 13 maintains the imaging unit 22 of endoscope 20 unblocked, though somewhat restricting the Field Of Illumination (FOI) and the Field Of View (FOV). In other embodiments, balloon 13 need not protrude from the distal end of endoscope 20, but rather reach the same plane as the plane of the distal end of endoscope 20, thereby not restricting neither the FOI nor the FOV of endoscope 20.

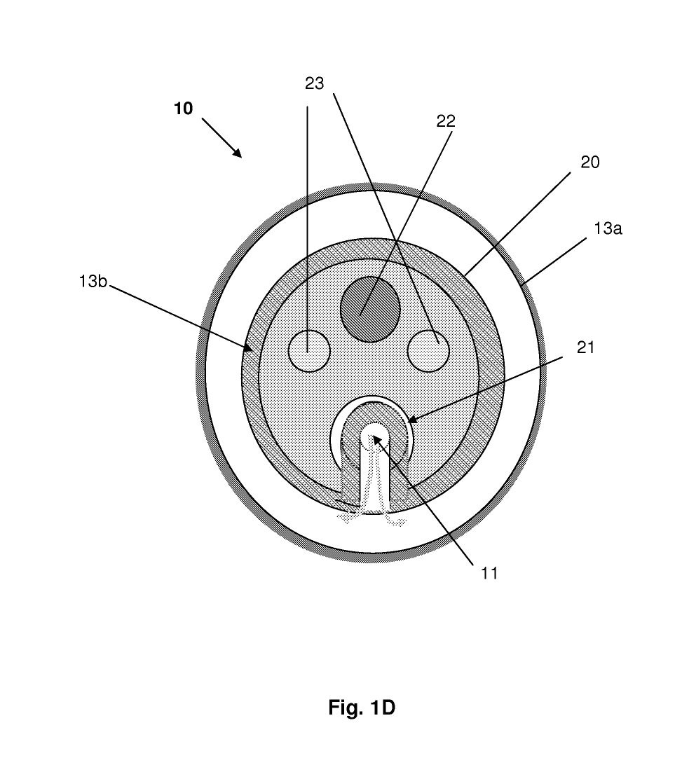

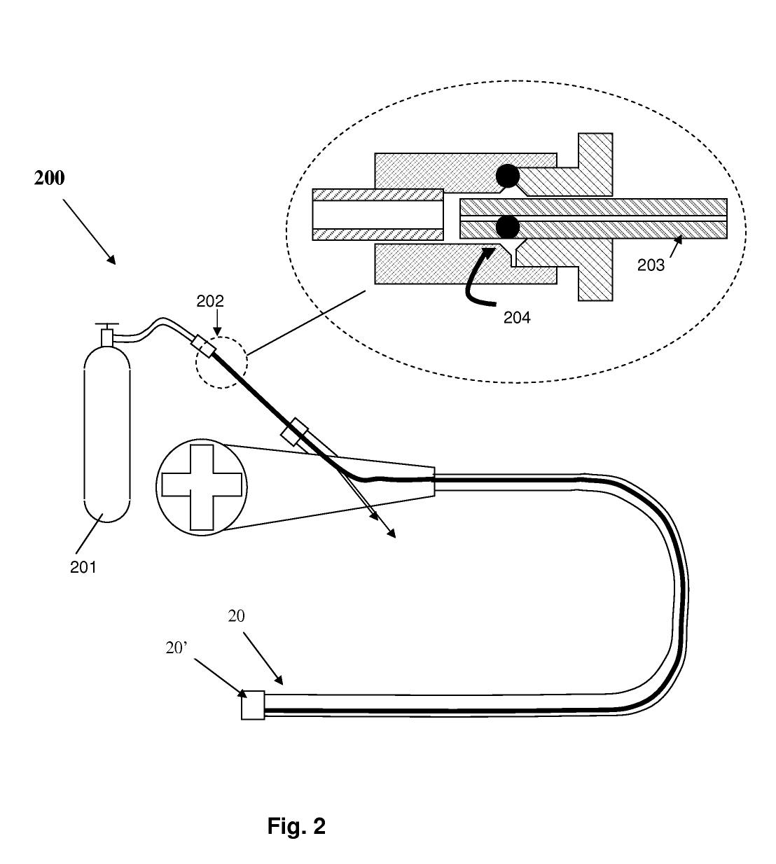

Reference is now made to FIG. 2, which schematically illustrates a cryotherapy system in accordance with one embodiment of the present invention. More specifically, FIG. 2 illustrates the connection of the proximal end of the cryotherapy device to a pressurized tank, in embodiments where the cryotherapy device is inserted through the distal end of an endoscope. FIG. 2 illustrates cryotherapy system 200 which may comprise an endoscope 20 through which a cryotherapy device 20′ may pass through. The cryotherapy device 20′ attached to endoscope 20 may be similar to cryotherapy device 10 as illustrated in FIGS. 1A-C.

System 200 may further comprise a high pressure tank 201, which may comprise a cryogenic fluid and keep it stored at a high pressure. High pressurized fluid, when exiting through a nozzle or orifice while kept insulated so that no heat is exchanged with the environment, cools to a lower temperature according to Joule-Thomson effect, i.e., a decrease in fluid pressure may cause a decrease in fluid temperature. The final fluid temperature, after the fluid exits the nozzle, should be suitable for cryosurgery treatment.

Cryotherapy device 20′ may be attached to high pressure tank 201 through connector 202. A connector 202, which may connect the proximal end of cryotherapy device 20′ to a pressurized tank 201, is needed when the cryotherapy device 20′ is inserted through the distal end of endoscope 20, as described in some embodiments of the present invention. When the cryotherapy device is inserted through the endoscope's distal end, there is no need for any connecting means at the device's distal end, but rather a need for connecting means at the proximal end of the cryotherapy device. In some embodiments, connector 202 may comprise an O-ring which may hold the tube of the high pressure tank 201 and the high pressure tube of the cryotherapy device 20′ together. Other means of attaching the high pressure tube of the cryotherapy device 20 to the high pressure tank 201 may be used.

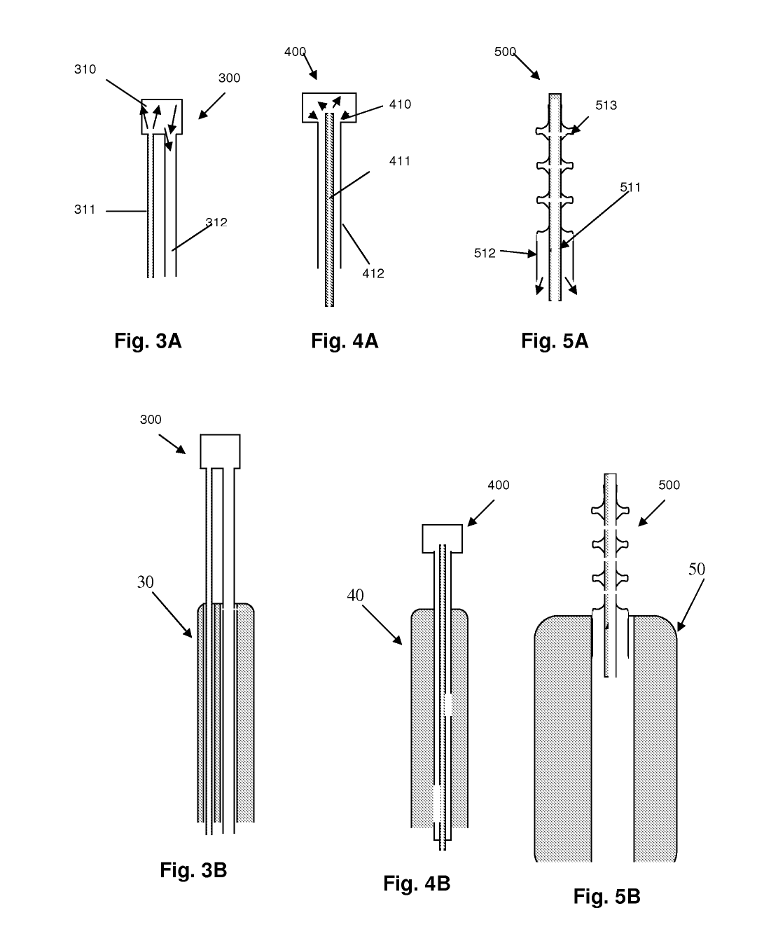

Reference is now made to FIGS. 3A and 3B, which illustrate schematic lengthwise sectional views of a cryotherapy device's distal end before and during insertion through an endoscope's distal end, respectively, in accordance with one embodiment of the present invention. FIGS. 3A-B illustrate a cryotherapy device, which may define a closed system as similarly defined in cryotherapy device 10 which is described in FIG. 1. A closed system means that no cryogenic fluid exits the cryotherapy device to be in direct contact with the tissue, but rather the fluid is confined to the boundaries of a cooling member, i.e., to the boundaries of either the balloon 13 walls, or to the boundaries of cooling finger 310 as described in FIGS. 3A-3B, and causes the tissue to freeze by cooling the balloon 13 or the cooling finger 310.

FIG. 3A illustrates the cryotherapy device 300 before insertion through an endoscope 30, while FIG. 3B describes how cryotherapy device 300 is inserted into endoscope 30 through the distal end of endoscope 30. As explained above with regard to FIG. 1, there are many advantages in inserting the cryotherapy device to the endoscope via the endoscope's distal end; such advantages apply here as well.

According to some embodiments, cryotherapy device 300 may comprise two tubes: one is a high pressure feed tube 311, which is the tube through which cryogenic fluid enters the device 300, and a second tube is a low pressure evacuation tube 312, which is the tube through which expanded cryogenic fluid exits device 300, in order to maintain a low temperature in device 300. According to some embodiments, cryotherapy device 300 may be inserted through an endoscope that includes two working channels, such that tube 311 is passed through one working channel, while tube 312 is passed through a second working channel.

In some embodiments, at the distal end of device 300 is a cooling finger 310. Cooling finger 310 may have a shape similar to that of a spoon, which may be curved so as to fit into cylindrically shaped lumens, and be able to touch only a specific area of the cylindrical lumen, and not touch the entire inner circular boundary of the lumen, as does balloon 13 (FIG. 1). Typically, a cooling finger may be applied when there is a need to treat a restricted area of the lumen wall's tissue, and not the entire lumen wall's inner circular boundary.

According to some embodiments, the size of cooling finger 310 may be dictated by the lumen it is to enter, e.g., for treatment of esophageal or small bowel tissue, cooling finger 310 may have one size, while for treatment of colon tissue, cooling finger 310 may have a larger size, since the colon's diameter is larger than the diameter of the esophagus and than the diameter of the small bowel. In other embodiments, one cooling finger size may be used for treatment of the various GI tract organs, and when necessary, the cooling finger may be twisted and turned such that its rounded edge may touch and freeze more than one area of interest.

In some embodiments, the cooling finger 310 may be made of a biocompatible metal, e.g., stainless-steel medical grade, titanium foil and others (including coated or surface treated alloys). In other embodiments, cooling finger 310 may be made of various polymers that have a thin wall in order to quickly transfer the low coolant's temperature to the area of interest, while being hard enough so as to not change its shape due to the high pressure at which the coolant enters into it. In other embodiments, the cooling finger 310 may be made of elastic materials and may thus have an adjustable shape, which may be changed and adjusted according to an area to be treated. For example, cooling finger 310 may be made of bio-grade polyurethane, Polyethylene terephthalate (PET) or nylon elastomers. In other embodiments, other materials may be used.

Reference is now made to FIGS. 4A and 4B, which illustrate schematic lengthwise sectional views of a cryotherapy device's distal end before and during insertion through an endoscope's distal end, respectively, in accordance with another embodiment of the present invention. FIGS. 4A-B illustrate a cryotherapy device 400 which may comprise a cooling finger 410 similar to cooling finger 310, as described in FIGS. 3A and 3B. However, unlike device 300, which may comprise two separate tubes for cooling fluid feed and evacuation, device 400 may comprise two concentric tubes.

In some embodiments, cryotherapy device 400 may comprise a high pressure feed tube 411 through which high pressurized fluid enters the cooling finger 410, and a low pressure evacuation tube 412 through which expanded fluid may exit the cooling finger 410. In some embodiments, the high pressure tube 411 may pass through low pressure tube 412, such that the two tubes are concentric. Typically, the high pressure tube is the one passing through the low pressure tube, since the high pressure tube typically has a small diameter in order to keep the fluid pressurized (the lower the volume for fluid to flow in, the higher its pressure is), while the low pressure tube is typically of a larger diameter in order to maintain a lower pressure (the more volume the fluid has to flow in, the lower the fluid's pressure is). Cryotherapy device 400 may be inserted through an endoscope's 40 working channel, as illustrated in FIG. 4B.

Reference is now made to FIGS. 5A and 5B, which illustrate schematic lengthwise sectional views of a cryotherapy device's distal end before and during insertion through an endoscope's distal end, respectively, in accordance with yet another embodiment of the present invention. FIGS. 5A and 5B illustrate a cryotherapy device 500, which may be inserted through an endoscope 50 working channel. Device 500 may comprise a high pressure tube 511, through which cryogenic fluid enters device 500, and a low pressure tube 512, which may be used to vent and evacuate fluid that was expanded after its exit through pressurized tube 511. According to some embodiments, the high pressure tube 511 and low pressure tube 512 may be concentric, e.g., high pressure tube 511 may pass through low pressure tube 512. Device 500 may further comprise an expandable balloon 513, which may expand upon coolant's entry. Cryogenic fluid may enter the deflated balloon 513 (FIG. 5A) and thus cool it and inflate it to reach a predetermined diameter. The final diameter of the expandable balloon 513 may be determined such that it fits the diameter of the various GI tract organs, e.g., esophagus, small intestine (small bowel) and the large intestine (colon).

In some embodiments, high pressure tube 511 may comprise one or more openings through which coolant may enter the balloon 513 in order to cool and inflate it. As illustrated in FIG. 5A, high pressure tube 511 may comprise a plurality of openings, which are located at a short distance from one another, along the longitudinal axis of high pressure tube 511. When cryogenic fluid is pressurized through high pressure tube 511, the fluid may exit tube 511 through its plurality of openings and thus inflate the balloon 513 sequentially, in an accordion-like motion, i.e., fluid first exits the openings that are located proximally to the operator of device 500, and then exits through the more distally located openings thus creating an accordion-like motion of inflation.

According to some embodiments, the balloon 513 may be made of similar materials as the materials balloon 13 (FIG. 1) is made of, e.g., latex, bio-grade polyurethane, Polyethylene terephthalate (PET) or nylon elastomers.

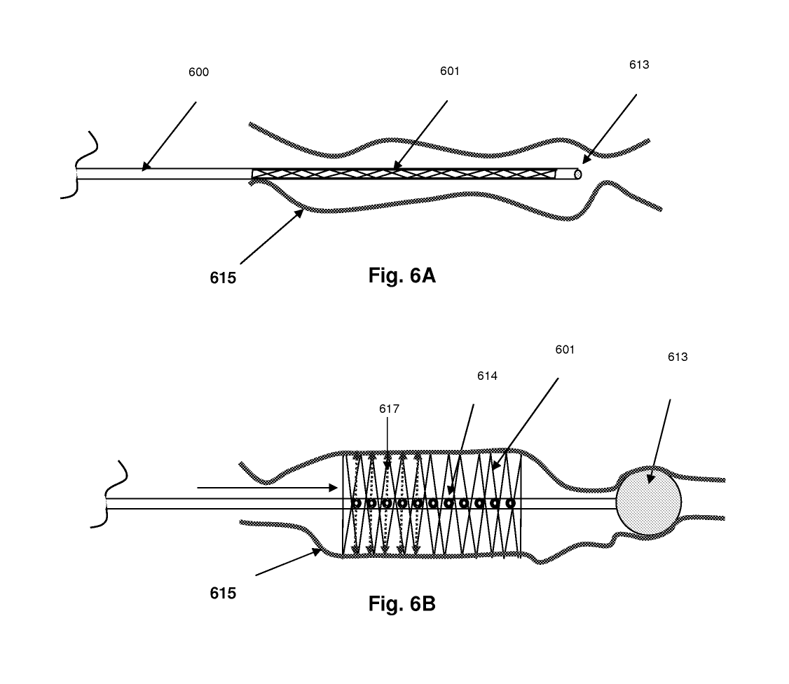

Reference is now made to FIGS. 6A and 6B, which illustrate schematic lengthwise sectional views of a cryotherapy device before and after immobilization to the lumen, respectively, in accordance with one embodiment of the present invention. Catheter 600 may be inserted through an endoscope but may also be passed through other in-vivo devices, or may not pass through any additional device. When inserted through an endoscope, catheter 600 may have attached a mesh 601, which may initially be in a folded or collapsed configuration (FIG. 6A). In some embodiments, catheter 600 may further comprise a balloon 613, which may be attached at the catheter 600 distal end. According to some embodiments, the balloon 613 may be inserted through an endoscope in a deflated configuration (FIG. 6A).

Once fluid is pressurized through catheter 600, the fluid may inflate balloon 613, thus causing it to change its configuration from a deflated state (FIG. 6A) to an inflated state (FIG. 6B). Subsequent to the balloon's inflation, an operator may open mesh 601 to its unfolded configuration (FIG. 6B). In some embodiments, the pressurized fluid may cause mesh 601 to unfold. According to some embodiments, unfolded mesh 601 may assist in pushing the lumen walls at a predetermined distance from catheter 600. Thus, once cryogenic fluid is further pressurized through catheter 600, it may exit through a plurality of openings or nozzles 614, and spray 617 may be homogenously sprayed all around the now cylindrically shaped lumen walls. In some embodiments, mesh 601 may hold the catheter 600 such that it is substantially concentric with the lumen walls surrounding it, thus the spray 617 may substantially have the same effect on any tissue that is located around openings 614. By unfolding mesh 601, which may thus push the cylindrical lumen wall such that it is substantially concentric with catheter 600, the operator may ensure that every section of the cylindrically shaped lumen wall is sprayed by substantially the same amount of fluid for the same time period.

According to some embodiments, balloon 613 may ensure that cryogenic fluid, which typically expands to gas after being pressurized out of catheter 600, is blocked by balloon 613. Balloon 613 may prevent expanded fluid from leaving the area being treated and entering other GI regions. For example, when catheter 600 is inserted into the esophagus 615 in order to treat esophageal lesions, balloon 613 may prevent coolant from going past the esophagus 615 and reaching the stomach. If cryogenic fluid (typically gas) reaches the stomach, the cryogenic fluid may cause the stomach to inflate, which may harm the stomach by, for example, perforating it. Therefore, balloon 613 may be designed to inflate such that it blocks passage of fluid or gas into other GI organs, where gas may cause harm. Balloon 613 may inflate such that its entire circumference is in contact with the lumen wall, thus preventing leakage of fluid or gas past the treated area. In some embodiments, there may further be means for evacuating expanded fluid from the treated area.

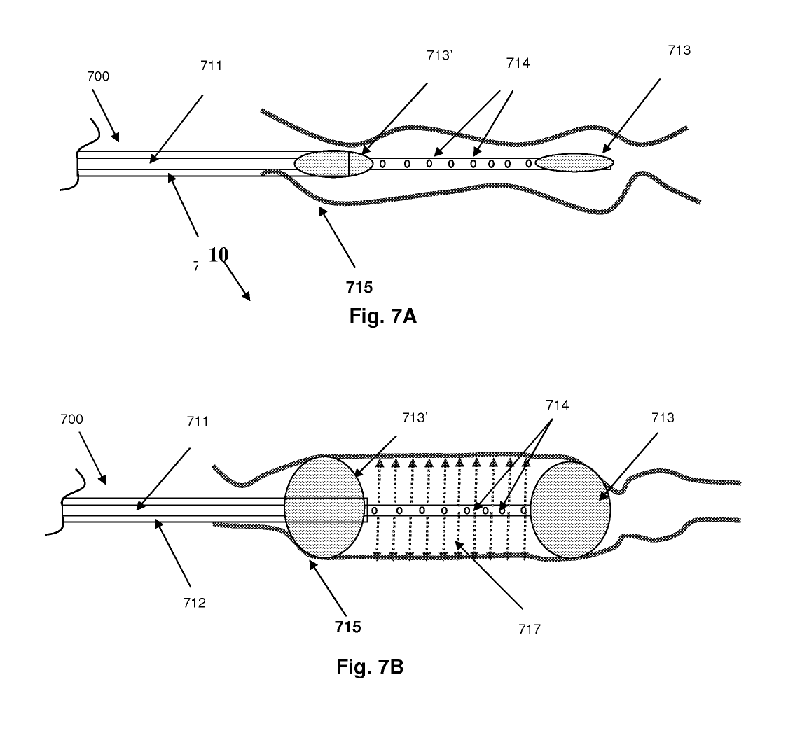

Reference is now made to FIGS. 7A and 7B, which illustrate schematic lengthwise sectional views of a cryotherapy device before and after immobilization to the lumen, respectively, in accordance with another embodiment of the present invention. According to some embodiments, a catheter 700 may be inserted through an endoscope, while in other embodiments it need not pass through an endoscope, but may be passed through other in-vivo devices, or may not be passed through any additional device.

In some embodiments, catheter 700 may comprise two tubes: tube 711 is for pressurized incoming cryogenic fluid, which may pass through a second tube 712 for low pressure outgoing fluid. Typically tubes 711 and 712 are concentric. Incoming cryogenic fluid tube 711 may be longer than the lower pressure tube 712, and may comprise a plurality of nozzles or orifices 714 through which coolant may be pressurized in order to freeze in-vivo lesions. Typically, openings or nozzles 714 may be positioned along the circumference of tube 711, so as to allow fluid to be sprayed all around the lumen wall that surrounds the tube 711(illustrated as spray 717).

In some embodiments, tube 711 may further comprise a balloon 713, which may be similar to balloon 613 (illustrated in FIGS. 6A-B). Balloon 713 may be located at tube 711 distal end. In its initial state, balloon 713 may be in a deflated configuration (FIG. 7A), whereas, following fluid flow into the balloon 713, it may inflate (FIG. 7B) such that it may block passage of fluid past it. In some embodiments, catheter 700 may be inserted through the esophagus 715, and thus similarly to balloon 613, balloon 713 may prevent expanded cryogenic fluid (typically gas) from entering the stomach.

According to some embodiments, tube 711 may comprise a second balloon 713′, which may be located closer to the proximal end of tube 711. In some embodiments, balloons 713 and 713′ may be located at both ends of the openings 714 of tube 711, such that the openings 714 may be located in between balloon 713 and balloon 713′. Balloon 713′ may assist in confining the coolant's expansion to in between balloons 713 and 713′, thus avoiding leakage of expanded fluid to neither a proximal nor distal location along the GI.

In some embodiments, fluid may be pressurized through tube 711 and may first inflate balloons 713 and 713′ and only later exit through nozzles 714. In some embodiments, the openings of each of balloon 713 and of balloon 713′ through which the coolant may enter into the balloons may be of a larger diameter than the diameter of nozzles 714 (which should typically be of a small diameter in order to cause fluid to exit at high pressure). Therefore, there may be less resistance in fluid flowing into balloons 713 and 713′ than fluid flowing into nozzles 714, thus fluid may first fill balloons 713 and 713′ and only then exit through nozzles 714.

According to other embodiments, balloon 713 and/or balloon 713′ need not be inflated by the cryogenic fluid, but rather may be inflated by other means, for example, the balloons may be filled with liquids such as water or saline, or may be filled with air. The liquids or air may be passed through a tube passing along catheter 700 and reaching the balloons' openings. In such embodiments, there is no need to compromise between the amount of fluid, pressure or other parameters that are required for inflating the balloons and the amount of fluid, pressure or other parameters that are required to treat a lesion.

In some embodiments, since catheter 700 may comprise an evacuation tube 712 in addition to an incoming cryogenic fluid tube 711, it may sufficiently evacuate expanded cryogenic fluid from within the treated lumen and its confined surroundings.

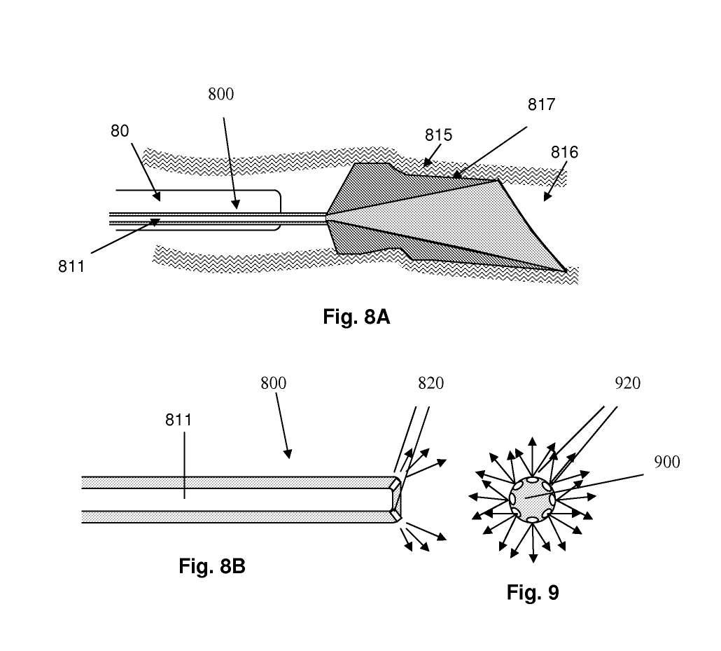

Reference is now made to FIGS. 8A and 8B, which illustrate lengthwise sectional views of a further cryotherapy device in accordance with one embodiment of the present invention. In current cryotherapy devices, which involve spraying an area of interest with cryogenic fluid, in order to freeze and thus treat an area of interest, the jet is directed at a forward direction, parallel to a forward moving direction of the endoscope carrying the cryotherapy device. According to embodiments of the present invention, as illustrated in FIGS. 8A and 8B, cryotherapy device 800 may be passed through endoscope 80. Cryotherapy device 800 may comprise a high pressure tube 811 through which the pressurized coolant may pass. Cryotherapy device 800 may further comprise openings or nozzles 820 through which the cryogenic fluid exits the tube 811 and comes in direct contact with tissue 815. Cryotherapy device 800 may be a device which sprays the cryogenic fluid onto the tissue 815; such that some of the sprayed tissue is the treated area 817, which may comprise diseased tissue, while the rest of the sprayed area may be healthy tissue. Although no harm is done when healthy tissue is frozen, since the tissue is rejuvenated within several weeks, it is desirable to freeze as little healthy tissue as possible.

As illustrated in FIG. 8B, cryotherapy spray device 800 may comprise openings or nozzles 120 located on the sides of the device 800, e.g., openings 820 may be located at a direction substantially perpendicular to the forward moving direction of endoscope 80. Other angled directions, at which nozzles 820 may be located, may be used. In order for cryotherapy device 800 to easily spray the lumen wall, which is typically perpendicular to the forward moving direction of the endoscope, openings 820 may be located such that they are substantially parallel to the lumen wall tissue. Typically, device 800 comprises at least two openings, though more than two openings may be used.

According to FIG. 9, which illustrates an end view of a cryotherapy device in accordance with another embodiment of the present invention, cryotherapy device 900 may comprise a plurality of openings or nozzles 920. Openings 920 may be located on the circumference of device 900 (which may be similar to device 800 illustrated in FIGS. 8A-B) such that they spray the cryogenic fluid in a radial direction.

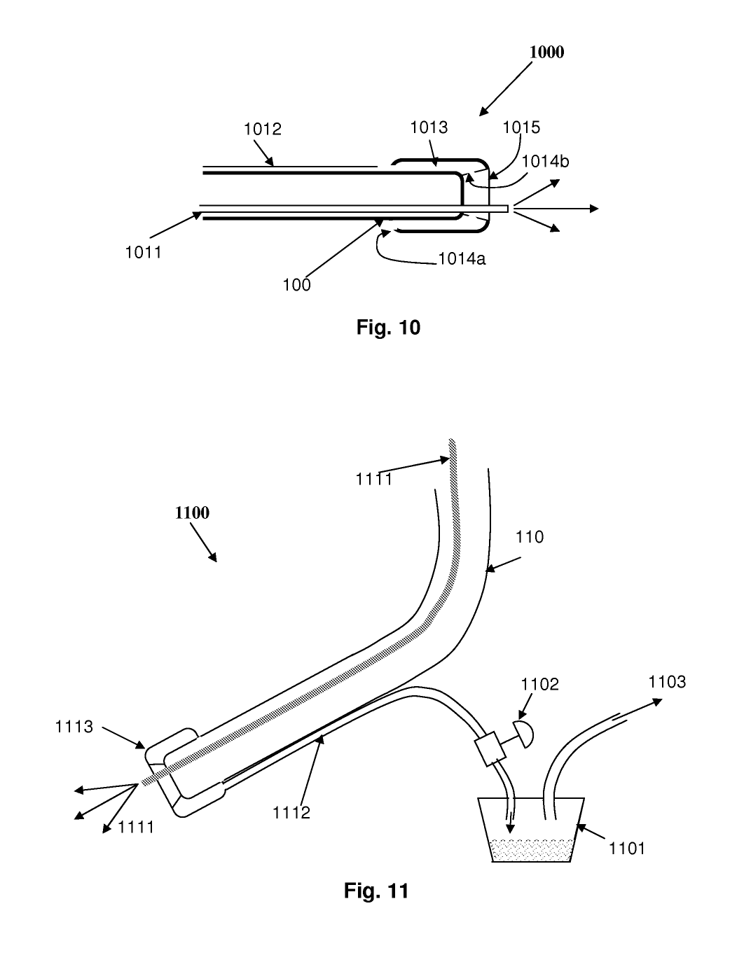

Reference is now made to FIG. 10, which illustrates a schematic lengthwise sectional view of a cryotherapy device's distal end in accordance with one embodiment of the present invention. According to some embodiments, cryotherapy device 1000, which is illustrated in FIG. 10, may be passed through endoscope 100, such that pressurized coolant tube 1011 may pass through the working channel of endoscope 100. Once the cryogenic fluid exits the pressurized tube 1011, the fluid expands and may freeze an area of interest with which the fluid comes in contact. In some embodiments, the suction or evacuation tube 1012 may pass along the circumference of endoscope 100, thus allowing a larger volume of expanded fluid to be evacuated from device 1000, since the diameter of evacuation tube 1012 is not restricted to the working channel's diameter.

According to some embodiments, device 1000 may further comprise a suction cup 1013, which may be placed over the distal end of endoscope 100. Suction cup 1013 may comprise suction ports 1014a, which may be located on the back end of cup 1013. That is, suction ports 1014a may be located closer to the proximal end of endoscope 100 and not closer to the front end of cup 1013, which is located closer to the distal end of endoscope 100. Locating suction ports 1014a on the back end of cup 1013 may enable suction of expanded fluid, while avoiding direct suction of tissue into evacuation tube 1012, which might harm the tissue. If suction ports 1014a would be located on the front end of suction cup 1013, tissue in close proximity to the suction ports might be sucked into the ports; however, when the suction ports 1014a are located at the back end of cup 1013, there is less chance of tissue getting sucked into the evacuation tube 1012.

In some embodiments, suction cup 1013 may comprise additional suction ports, e.g., suction ports 1014b. Suction ports 1014b may be located closer to the front end of cup 1013 than to its back end. However, in order to prevent direct contact between the suction ports 1014b and the tissue, such that tissue would not be sucked into evacuation tube 1012 through the suction ports 1014b, suction cup 1013 may comprise a protective grille 1015. Protective grille 1015 may be attached to cup 1013 so as to cover its front end, while distancing suction ports 1014b from the tissue. Protective grille 1015 may comprise holes through which pressurized coolant tube 1011 may be pushed in order to freeze an area of interest. Furthermore, protective grille 1015 may comprise holes through which expanded coolant may be sucked through suction ports 1014b. However, protective grille 1015 may prevent tissue from being sucked into suction ports 1014b, since it pushed the tissue away from ports 1014b, and its holes may be designed to not be large enough for sucking tissue there through.

Reference is now made to FIG. 11, which schematically illustrates a cryotherapy system in accordance with one embodiment of the present invention. The cryotherapy system as illustrated in FIG. 11 may comprise a cryotherapy device 1100 inserted through the working channel of endoscope 110. The cryotherapy device 1100 of FIG. 11 may be similar to the cryotherapy device 1000 as illustrated in FIG. 10, such that is may comprise a cryogen capillary (or pressurized coolant tube) 1111, through which the cryogenic fluid may pass before reaching the area of interest. In addition, the cryotherapy device 1100 may comprise a suction tube 1112, through which fluid may be evacuated from within the lumen to outside of the lumen. Suction tube 1112 may pass along the circumference of endoscope 110, and thus it is not limited to the working channel's diameter and may evacuate large volume of fluid within a relatively short period of time. The cryotherapy device 1100 as illustrated in FIG. 11 may further comprise a suction cup 1113, which may be similar to suction cup 1013 (FIG. 10) and may comprise similar suction ports (as suction ports 1014a, 1014b) and protective grille (as grill 1015).

According to some embodiments, the cryotherapy device 1100 may be attached, through evacuation tube 1112, to a vacuum suction setup, which may comprise a vacuum container 1101 that accumulates liquids. In some embodiments, vacuum container 1101 may comprise a tube for suction of liquid from the container 1101, thus causing lower pressure within evacuation tube 1112. A suction valve 1102 may preferably be positioned along suction tube 1112 prior to the entrance of suction tube 1112 into vacuum container 1101. Locating the suction valve 1102 before the vacuum container 1101 may assist the operator in stopping suction substantially immediately when backpressure accumulates within the lumen. If the suction valve 1102 would have been located after vacuum container 1101, e.g., along suction tube 1103, the suction wouldn't have stopped immediately after the operator closed the suction valve 1102, but rather the operator would have had to wait until pressure is reduced through the tube 1103, then through container 1101 and through suction tube 1112. In other embodiments, valve 1102 may be located after vacuum container 1101, e.g., along suction tube 1103. Closure of valve 1102 may then stop suction, though not immediately subsequent to the closure of valve 1102.

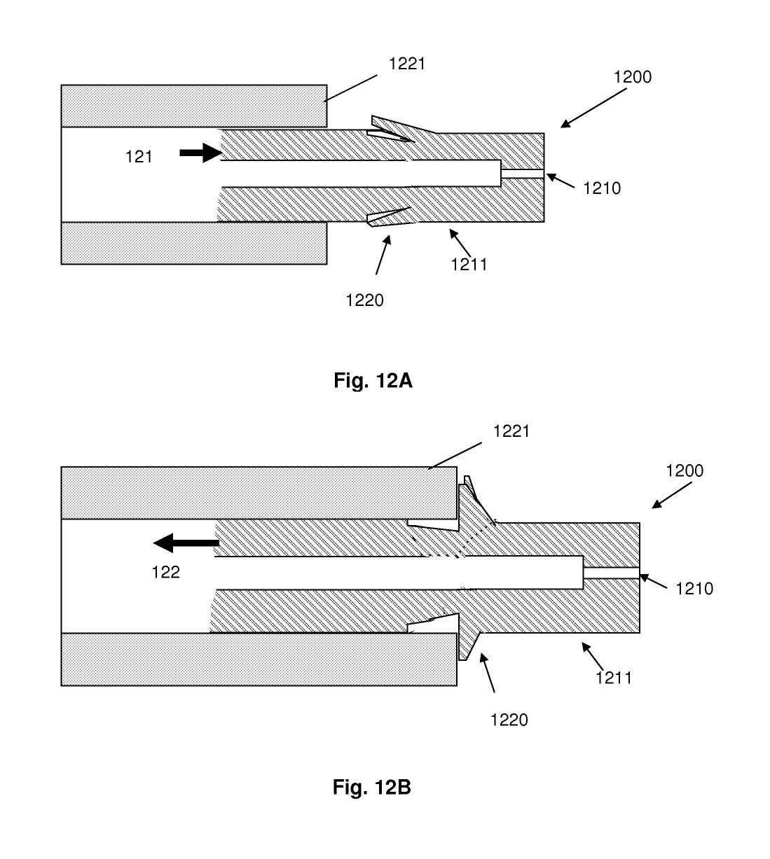

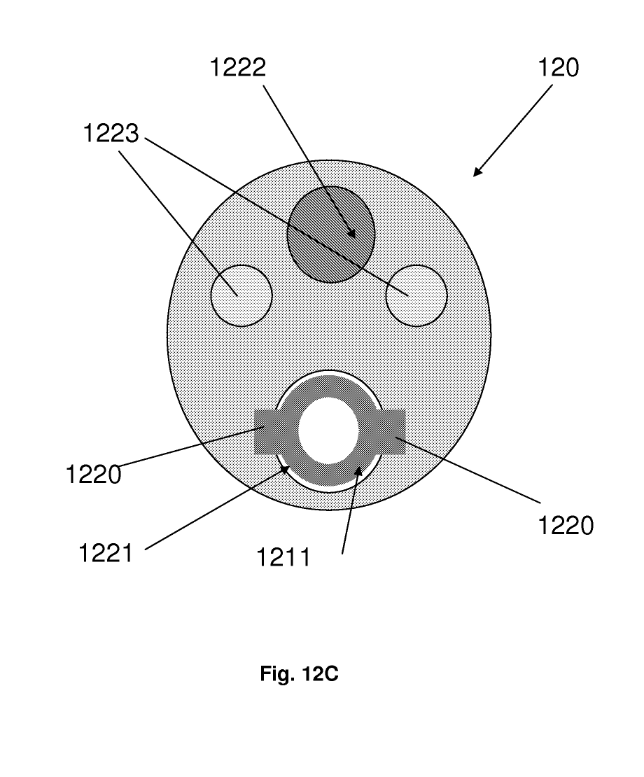

Reference is now made to FIGS. 12A and 12B, which illustrate schematic lengthwise sectional views of a cryotherapy device's distal end before and after insertion through an endoscope's distal end, respectively, in accordance with one embodiment of the present invention. A cryotherapy device 1200, according to embodiments of the present invention, may comprise a pressurized coolant tube 1211 through which cryogenic fluid may pass until it exits through nozzle 1210. Nozzle 1210 typically has a reduced diameter than the diameter of tube 1211, in order to spray the cryogenic fluid at a high pressure onto a tissue to be treated or to enable a Joule Thompson effect to occur. In addition, nozzle 1210, typically having a diameter smaller than the diameter of tube 1211, may assist in controlling the amount of sprayed coolant. Tube 1211 may be inserted through a working channel 1221 of an endoscope 120 (FIG. 12C), and thus does not obstruct the imaging and/or illuminating channels of endoscope 120.

Cryotherapy device 1200 may further comprise wings 1220, which may be attached onto tube 1211. According to some embodiments, wings 1220 may protrude by only a few millimeters or fractions of millimeters from the outer diameter of tube 1211, which may not affect an easy insertion of the tube 1211 through the working channel 1221. An operator may push the tube 1211 through the working channel 1221 towards the endoscope's distal end, e.g., in the direction illustrated as arrow 121, until the wings 1220 are entirely pushed outside of the opening of working channel 1221. When the tube 1211 is pushed through the endoscope, the wings 1220 may be in a folded configuration (FIG. 12A).

Subsequent to pushing the tube 1211 through the working channel 1221, such that wings 1220 may be pushed outside of the opening of working channel 1221, the operator may begin to pull the tube 1211 towards the proximal end of the endoscope, i.e., in the direction illustrated by arrow 122. Once the wings 1220 reach the opening of working channel 1221, the wings 1220 are forced to open and thus change their configuration from folded to unfolded (FIG. 12B). When the wings 1220 are in an unfolded configuration, they are pressed and lean against the opening of working channel 1221, thus causing tube 1211 to be tightly attached to the endoscope through which it passes. Furthermore, when wings 1220 are pressed against the opening of working channel 1221, tube 1211 may be prevented from freely rotating relative to the rotation of the endoscope 120 in addition to the location of nozzle 1210 being constant and known relative to the distal end of endoscope 120. Tube 1211 is then forced to rotate with the endoscope as one unit, which makes it easier on the operator to control movement and rotation of cryotherapy device 1200.

Reference is now made to FIG. 12C which illustrates a schematic upper view of the cryotherapy device of FIGS. 12A-B, in accordance with one embodiment of the present invention. According to FIG. 12C, the pressurized coolant tube 1211 may pass through working channel 1221 of endoscope 120. Wings 1220 are illustrated in their unfolded configuration and are pressed against the opening of working channel 1221. As shown in FIG. 12C, the cryotherapy device 1200 obstructs neither the imaging channel 1222 nor the illuminating channels 1223, thus enabling the operator to view the area of interest, while performing the cryosurgical procedure.

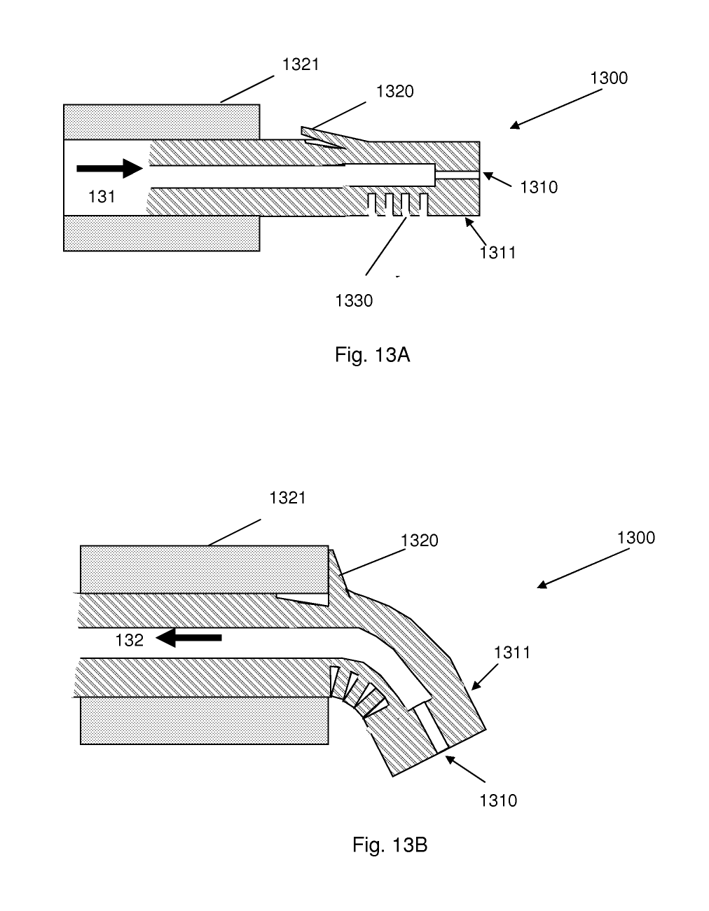

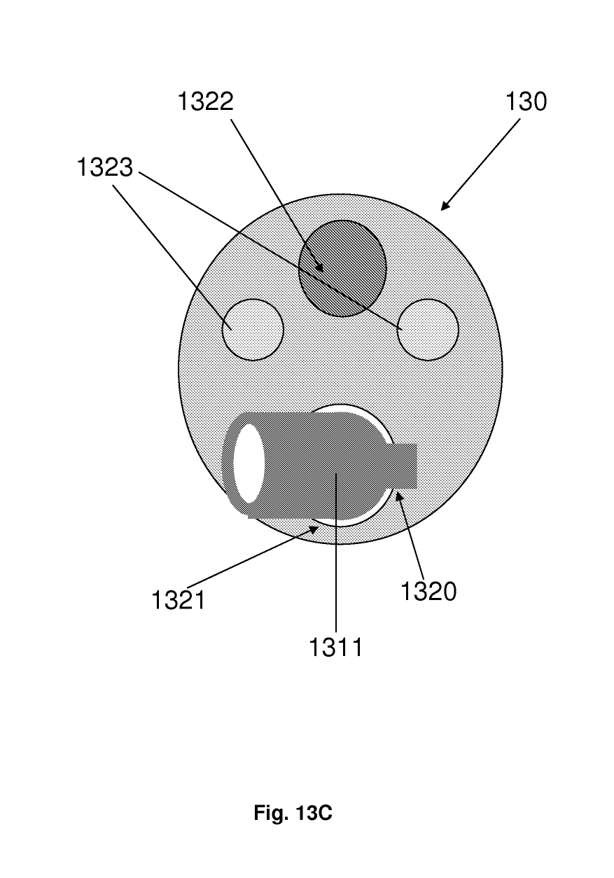

Reference is now made to FIGS. 13A and 13B which illustrate schematic cross sections of a cryotherapy device's distal end before and after insertion through an endoscope's distal end, respectively, in accordance with another embodiment of the present invention. A cryotherapy device 1300, according to embodiments of the present invention, may comprise a pressurized coolant tube 1311 through which cryogenic fluid may pass until it exits through nozzle 1310. Nozzle 1310 typically has a diameter smaller than the diameter of tube 1311, in order to allow the cryogenic fluid to be sprayed at a high pressure onto a tissue to be treated or to enable a Joule Thompson effect to occur. In addition, nozzle 1310, typically having a diameter smaller than the diameter of tube 1311, may assist in controlling the amount of sprayed coolant. Tube 1311 may be inserted through a working channel 1321 of an endoscope 130 (FIG. 13C), thus not obstructing the imaging and/or illuminating channels of endoscope 130.

Cryotherapy device 1300 may further comprise a wing 1320, which may be attached onto tube 1311. According to some embodiments, wing 1320 may protrude by only a few millimeters from the outer diameter of tube 1311, which may not affect the easy insertion of the tube 1311 through the working channel 1321. For example, wing 1320 may protrude from the outer diameter of tube 1311 by approximately 1 mm, such that inserting tube 1311 through the working channel 1321 would not raise any difficulties. An operator may push the tube 1311 through the working channel 1321 towards the distal end of endoscope 130, e.g., in the direction illustrated as arrow 131, until wing 1320 is entirely pushed outside of the opening of working channel 1321. When the tube 1311 is pushed through the endoscope, the wing 1320 may be in a folded configuration (FIG. 13A).

According to some embodiments, cryotherapy device 1300 may further comprise slots 1330. Slots 1330 may enable a bending motion by tube 1311.

Subsequent to pushing the tube 1311 through the working channel 1321, such that wing 1320 may be pushed outside of the opening of working channel 1321, the operator may begin to pull back the tube 1311 towards the proximal end of the endoscope, i.e., in the direction illustrated by arrow 132 (FIG. 13B). Once the wing 1320 reaches the opening of working channel 1321, the wing 1320 is forced to open and thus changes its configuration from folded to unfolded (FIG. 13B). When the wing 1320 is in an unfolded configuration, it is pressed against the opening of working channel 1321, thus causing tube 1311 to be tightly attached to the endoscope 130 through which it passes. Furthermore, when wing 1320 is pressed against the opening of working channel 1321, tube 1311 is prevented from freely rotating relative to the endoscope's rotation. Tube 1311 is then forced to rotate with the endoscope as one unit, which makes it easier on the operator to manipulate cryotherapy device 1300 and to direct it to any desirable direction.

In some embodiments, while tube 1311 is being pulled back by the operator, towards the proximal end of the endoscope (in the direction of arrow 132), in addition to wing 1320 being pressed against the opening of working channel 1321, slots 1330 may force tube 1311 to bend. Slots 1330, which are located along tube 1311, are typically located opposite the location of wing 1320, in order to achieve two functions when tube 1311 is pulled back towards the proximal end of endoscope 130. The first function may be achieved by wing 1320; wing 1320 may cause tube 1311 to be pressed against the opening of working channel 1321, so as to force tube 1311 to rotate along with endoscope 130 as one unit, once endoscope 130 is rotated by the operator. The second function may be achieved by slots 1330; slots 1330 may force the tube 1311 to bend, thus pointing the nozzle 1310 at a direction perpendicular to or angled with respect to a forward moving direction of endoscope 130, in order to apply easy side spraying on the lumen wall.

According to some embodiments, cryotherapy device 1300 may be able to point side ways (i.e., towards the lumen walls that are typically parallel to a forwards moving direction of endoscope 130). By rotating the endoscope through which device 1300 passes through, the operator may point nozzle 1310 to substantially any direction perpendicular to the lumen wall, thus enabling the operator to perform cryosurgery at almost any desirable location along the lumen wall.

Reference is now made to FIG. 13C which illustrates a schematic upper view of the cryotherapy device 1300 of FIGS. 13A-B, in accordance with one embodiment of the present invention. According to FIG. 13C, the pressurized coolant tube 1311 may pass through working channel 1321 of endoscope 130. Wing 1320 is illustrated in its unfolded configuration and may be pressed against the opening of working channel 1321, while tube 1311 may be bent towards slots 1330 (not shown), in a direction typically opposite the location of wing 1320. As shown in FIG. 13C, the cryotherapy device 1300 obstructs neither the imaging channel 1322 nor the illuminating channels 1323, thus enabling the operator to view the area of interest, while performing the cryosurgical procedure.

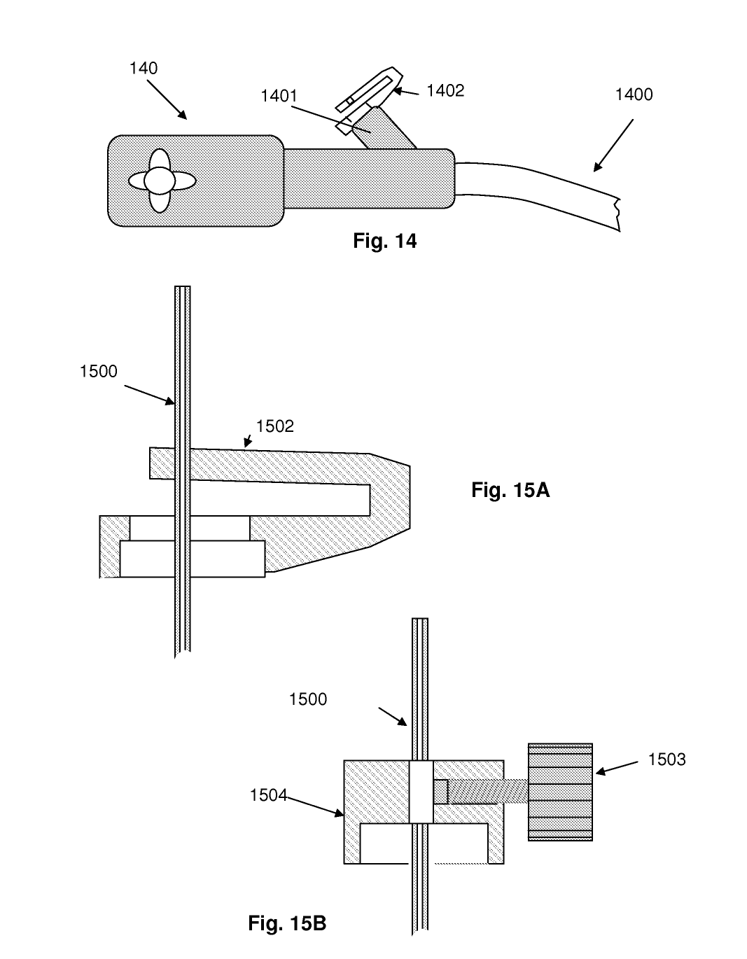

Reference is now made to FIG. 14, which illustrates a location along the endoscope at which are located securing means for attaching a cryotherapy device to an endoscope in accordance with one embodiment of the present invention. In order to ensure that a cryotherapy device 1400, which may be similar to device 1200 (FIGS. 12A-C) or to device 1300 (FIGS. 13A-C), is tightly attached to the endoscope 140 through which it passes, so as to rotate with the endoscope 140 as one unit, the cryotherapy device 1400 needs to be further attached to the endoscope 140 at port 1401, which is the entrance of cryotherapy device 1400 into the endoscope 140. In addition to wings (such as wings 1220 and 1320) which assist in pressing the cryotherapy device against the endoscope at the distal end of the endoscope 140, there is a need for additional securing means 1402, which may be located at the proximal end of endoscope 140.

Reference is now made to FIGS. 15A-15B, which illustrate schematic lengthwise sectional views of two attachment mechanisms for attaching a cryotherapy device to an endoscope, in accordance with other embodiments of the present invention. FIGS. 15A and 15B illustrate possible securing means which may be used for securing the cryotherapy device to the endoscope, at the proximal end of the endoscope, as illustrated in FIG. 14. According to FIG. 15A, a clamp 1502 may be used to attach cryotherapy device 1500 to an endoscope through which it passes. Clamp 1502 may be placed onto device 1500 following insertion of cryotherapy device 1500 through an endoscope.

In some embodiments, the cryotherapy device may be inserted through the endoscope's working channel and may be pushed towards the distal end of the endoscope. The cryotherapy device typically comprises an attachment means at the distal end of the device, e.g., wings 1220 or 1320, such that once the device 1500 is pulled back towards the proximal end of the endoscope, the device 1500 is pressed against the distal end of the endoscope. In order to secure the proximal end of the device 1500 to the endoscope in order to prevent the endoscope from sliding forward (towards distal end) after being pulled back (towards proximal end) by the operator of the device 1500, a clamp 1502 may be applied on device 1500 at the proximal end of the device 1500, near the entrance to the endoscope's working channel, which is the entrance through which device 1500 enters the endoscope. In some embodiments, clamp 1502 may be a self-locking clamp.

According to FIG. 15B, other means of attachment may be used for securing the cryotherapy device 1500 to the endoscope at the proximal end of the endoscope, in order to prevent the cryotherapy device 1500 from sliding forward (towards distal end) after being pulled back (towards proximal end) by the operator. For example, a lock screw 1503 may be screwed into cup 1504, which may be placed around the device 1500 near its entrance to the endoscope through which it passes, in order to tighten device 1500 to the endoscope. In some embodiments, screw 1503 may comprise a “v” grooved tip in order to avoid damage done to the tube of device 1500 when screwed into cup 1504. In other embodiments, instead of a screw and cup, an O-ring may be placed over the tube of cryotherapy device 1500, near the entrance of device 1500 into the endoscope's working channel. A nut may be screwed to press down on the O-ring in order to fasten cryotherapy device 1500 to the endoscope it passes there through.

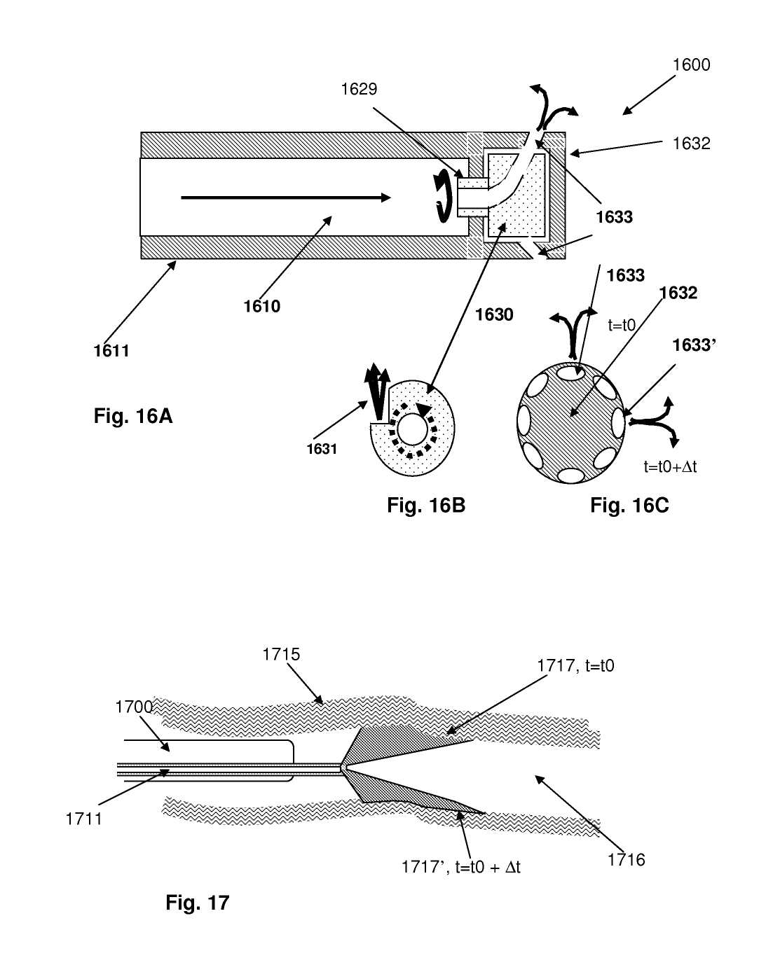

Reference is now made to FIGS. 16A to 16C. FIG. 16A illustrates a schematic lengthwise sectional view of a cryotherapy device, in accordance with one embodiment of the present invention, and FIGS. 16B and 16C illustrate cross sectional and schematic end views of components within and outside the cryotherapy device of FIG. 16A, respectively, in accordance with one embodiment of the present invention.

Cryotherapy device 1600 may comprise a pressurized tube 1611 through which cryogenic fluid 1610 may pass before reaching an area of interest. In some embodiments, cryotherapy device 1600 may further comprise a rotatable head 1629 which may be attached to rotatable circular component 1630. According to some embodiments, rotatable head 1629 may comprise a concentric hole through which cryogenic fluid 1610 may pass through after passing through tube 1611. The cryogenic fluid 1610 may further pass through rotatable component 1630, which may include at least one opening 1631 for the fluid to exit from and thus by sprayed onto the tissue of interest. Typically, the opening 1631 in rotatable circular component 1630 is positioned such that the tangential component of fluid 1610 that exits through opening 1631 may cause a free spin or rotation of component 1630 around a longitudinal axis of device 1600.

When circular component 1630 rotates due to the force at which fluid 1610 exits through opening 1631 of component 1630, fluid 1610 may be sprayed at 360 degrees, like a rotating-head sprinkler. In some embodiments, attached to the distal end of tube 1611 there may be a cover 1632, which may comprise a plurality of nozzles, e.g., openings 1633 and 1633′. In some embodiments, rotatable components 1629 and 1630 may rotate within tube 1611, while cover 1632 may ensure that the rotating components of cryotherapy device 1600 are not in direct contact with the tissue surrounding the device 1600, in order to avoid tissue getting caught inside rotatable components 1629 and/or 1630. Cover 1632 may comprise openings, such as openings 1633 and 1633′, but may comprise many more openings. The openings in cover 1632 may limit the amount of fluid that comes in contact with the area of interest, at any given moment during cryosurgical procedure, by having a smaller diameter than the diameter of opening 1631. In some embodiments, the openings in cover 1632 restrict the amount of fluid that exits from tube 1611 and may thus enable treatment of a tissue of interest, while avoiding over exposure of tissue to cryogenic fluid, and allowing sufficient evacuation of fluid during the cryosurgical procedure.

In some embodiments, the sprinkler-like cryotherapy device 1600 may enable peripheral treatment of the entire tissue that surrounds the distal end of cryotherapy device 1600, since cryotherapy device 1600 may rotate in 360 degrees and thus achieve full coverage of portions of cylindrically shaped lumens, e.g., the esophagus, small bowel and colon. As illustrated in FIG. 16C, during rotation of component 1630, cryogenic fluid may exit through opening 1633 at time t₀, and after a certain time lapse Δt, e.g. at t₀+ Δt the fluid may exit through opening 1633′, which is located at a distance from opening 1633. This way, coolant may be sprayed out of different openings at different times, during the rotation of rotatable component 1630, and may thus achieve full coolant coverage over tissue surrounding the distal end of device 1600.

According to some embodiments, device 1600 may be forced to rotate with the endoscope it passes through, as one unit, in order to ease manipulation and directionality of the device 1600 towards an area of interest, by means similar to the means illustrated in FIGS. 12A to 15B. However, the distal end of device 1600 may freely rotate as described above, in order to achieve peripheral treatment of tissue that surrounds the distal end of the device 1600.

Reference is now made to FIG. 17, which illustrates a schematic lengthwise sectional view of a cryotherapy device within a lumen, in accordance with one embodiment of the present invention. The cryotherapy device 1711 that may pass through endoscope 1700, may be similar to cryotherapy device 1600 (FIGS. 16A-16C), and may comprise a rotatable component that may cause the cryogenic fluid to be sprayed at 360 degrees around the distal end of device 1711. In some embodiments, the device 1711 may be pushed outside of endoscope 1700 such that device 1711 protrudes from the distal end of endoscope 1700, and may thus reach areas in lumen 1716 that are too narrow for the endoscope 1700 to enter. In other embodiments, device 1711 may be pushed out of the distal end of endoscope 1700 in order for the operator to be able to acquire images of the cryotherapy device 1711 during its operation of freezing tissue 1715.

After cryogenic fluid begins to flow through device 1711 at a pressurized manner, it may exit through openings located on a rotatable component that may be positioned at the distal end of device 1711 (e.g. components 1630 and corresponding opening 1631 in FIGS. 16A-16C) which may cause free spin of the rotatable component. When the rotatable component (e.g., component 1630) spins, coolant may be sprayed onto different area of the tissue during the rotation of the rotatable component around the longitudinal axis of device 1711. For example, coolant may first be sprayed onto tissue 1717 at time t₀, and then at time t₀+ Δt, the coolant may be sprayed onto tissue 1717′, which is located at a different section of the lumen 1716 surrounding device 1711. This technique of spraying different sections of the tissue at different time periods may enable coverage of substantial tissue area while reducing the need for extensive evacuation means, and may thus cause less trauma to the tissue and to the patient's body.

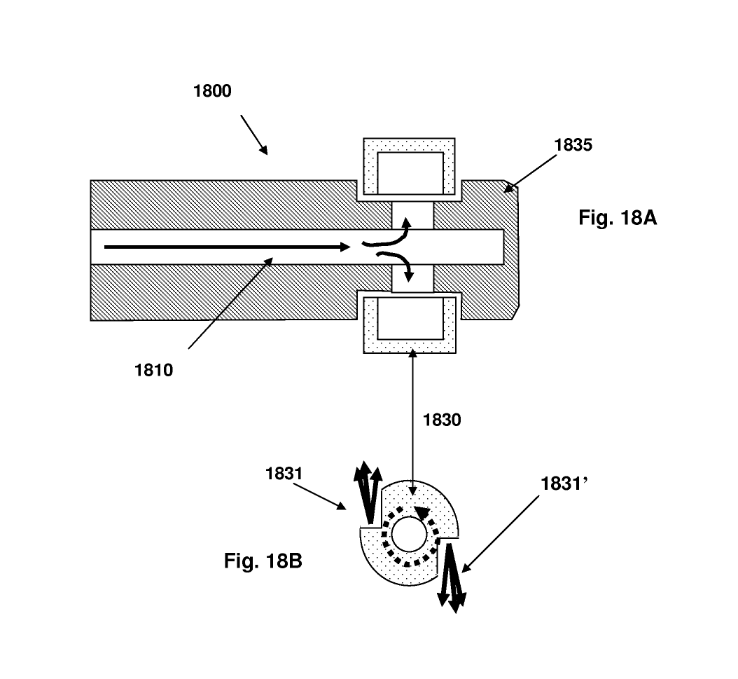

Reference is now made to FIGS. 18A and 18B. FIG. 18A illustrates a schematic lengthwise sectional view of a cryotherapy device, in accordance with another embodiment of the present invention, and FIG. 18B illustrates a schematic cross-sectional view of a component within the cryotherapy device of FIG. 18A, in accordance with an embodiment of the present invention.

Cryotherapy device 1800 may be inserted into the lumen through an endoscope, and pressurized cryogenic fluid 1810 may flow along and out of the device 1800 in order to be sprayed onto a tissue of interest. Device 1800 may comprise a rotatable component 1830, which may be located along the tube of device 1800, and which may be fastened to device 1800 by member 1835. During manufacturing of device 1800, rotatable component 1830 may be slid over the tube of device 1800 and member 1835 may be secured onto component 1830 by being, for example, thermally squeezed, screwed or glued onto it, so as to hold it in place and fasten it to device 1800.

According to some embodiments, rotatable component 1830 may comprise one opening 1831, while in other embodiments, component 1830 may comprise more than one opening, e.g., openings 1831 and 1831′. Other numbers of openings may be used. Once pressurized fluid 1810 is forced through device 1800, when the fluid reaches openings 1831 and 1831′, it may cause rotatable component 1830 to rotate. The force at which fluid 1810 is pushed outside of openings 1831 and 1831′ may cause a free spin or rotation of component 1830 around a longitudinal axis of device 1800. The tangential component of fluid 1810 that exits through the openings 1831 and 1831′ may cause rotation of component 1830 and may thus enable treatment of tissue that circles the distal end of device 1800.

According to some embodiments, device 1800 may be forced to rotate as one unit with the endoscope it passes through, by means similar to the means illustrated in FIGS. 12A-15B, in order to ease manipulation of the device 1800 and enable the operator to direct it towards an area of interest. However, the distal end of the device 1800 may be able to freely spin around a longitudinal axis of device 1800 in order to achieve peripheral treatment of tissue that surrounds the distal end of the device 1800.

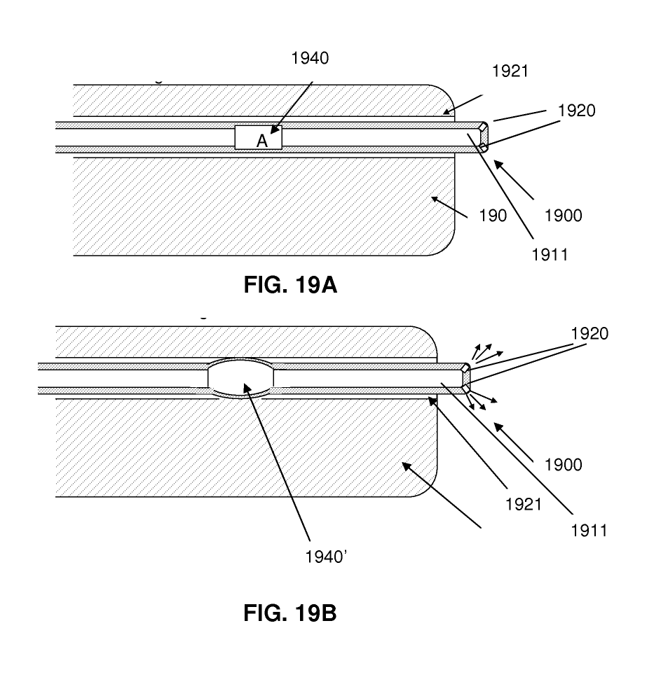

Reference is now made to FIGS. 19A and 19B which illustrate schematic lengthwise sectional views of a cryotherapy device, before and during operation, respectively, in accordance with an embodiment of the present invention. Cryotherapy device 1900 may be inserted through a working channel 1921 of endoscope 190 and may extend out of the distal end of endoscope 190. Cryotherapy device 1900 may comprise openings or nozzles 1920 for pressurizing and spraying the cryogenic fluid there through. According to some embodiments, cryotherapy device 1900 may further comprise an expandable section 1940, which may be in a deflated configuration (FIG. 19A) prior to coolant flowing through the pressurized coolant tube 1911 of device 1900.

During coolant flow through pressurized coolant tube 1911, expandable section 1940 may change its configuration to an inflated or expanded configuration 1940′ (FIG. 19B). When expandable section 1940′ is in its expanded configuration, it may cause the walls of pressurized coolant tube 1911 to be pressed against the walls of working channel 1921, thus forcing cryotherapy device 1900 to be tightly attached to endoscope 190. Expandable section 1940 may secure cryotherapy device 1900 to endoscope 190. Cryotherapy device 1900 may then be prevented from freely rotating relative to the rotation of endoscope 190, in addition to nozzles 1920 having a constant and known location relative to the distal end of endoscope 190.

The preceding specific embodiments are illustrative of the practice of the techniques of this disclosure. It is to be understood, therefore, that other expedients known to those skilled in the art or disclosed herein may be employed without departing from the scope of the following claims.

What is claimed is:

1. A cryotherapy device, which is passed through an endoscope, for treating gastrointestinal lesions, said device comprising:
a cooling member for contacting and treating a lesion;
a first tube for pressurizing cryogenic fluid there through and into the cooling member, wherein said cryogenic fluid is expanded after exiting said first tube; and
a second tube for evacuating said expanded cryogenic fluid from within the cooling member;
wherein said first tube is passed through an endoscope's working channel, and
wherein said second tube is passed along said endoscope's circumference.

2. The device according to claim 1, wherein said cooling member is a balloon.

3. The device according to claim 2, wherein said balloon is made of materials selected from a group consisting of: latex, bio-grade polyurethane, Polyethylene terephthalate (PET), and nylon elastomers.

4. The device according to claim 1 wherein said cooling member is a cooling finger.

5. The device according to claim 4, wherein said cooling finger is shaped to fit into cylindrically shaped lumens.

6. The device according to claim 4, wherein said cooling finger is made of materials selected from a group consisting of: stainless-steel medical grade, titanium foil, latex, bio-grade polyurethane, Polyethylene terephthalate (PET), and nylon elastomers.

7. A cryotherapy device, which is passed through an endoscope, for treating gastrointestinal lesions, said device comprising:
a first tube comprising at least one nozzle for pressurizing cryogenic fluid there through and onto a lesion within a lumen, wherein said cryogenic fluid is expanded after exiting said first tube;
a second tube for evacuating said expanded cryogenic fluid from within the lumen;
wherein said first tube is passed through an endoscope's working channel, and
wherein said second tube is passed along the circumference of the endoscope and
securing means attached to the first tube for securing the first tube to the endoscope's working channel.

8. The device according to claim 7, wherein said securing means comprises wings pressing against the opening of the endoscope's working channel.

9. The device according to claim 7, wherein said securing means comprises an expandable section located along the first tube for expanding during cryogenic fluid flow through said first tube.

10. The device according to claim 7, wherein said first tube comprises slots for enabling a bending motion by said first tube.

11. The device according to claim 7, further comprising a rotatable component for enabling free spin of the distal end of the device around a longitudinal axis of the device.
